(19) United States
(12) Patent Application Publication
(10) Pub. No.: US 2025/0281103 A1
(43) Pub. Date: Sep. 11, 2025

(54) APPARATUS AND METHODS FOR ACTIVE-FEEDBACK

(71) Applicant: BRAINTRAIN2020 LIMITED, Sheffield South Yorkshire (GB)

(72) Inventors: Richard MILLS, Sheffield South Yorkshire (GB); Maan VAN DE WERKEN, Sheffield South Yorkshire (GB)

(21) Appl. No.: 18/863,620

(22) PCT Filed: Mar. 3, 2023
(86) PCT No.: PCT/GB2023/050507

§ 371 Date: Nov. 6, 2024

(30) Foreign Application Priority Data
May 6, 2022 (GB) 2206686.4

(51) Int. Cl. A61B 5/00; A61B 5/16; A61M 21/00; A61M 21/02; G16H 40/63

(52) U.S. Cl. CPC A61B 5/4088; A61B 5/162; A61B 5/4809; A61B 5/4812; A61B 5/7405; A61B 5/742; A61B 5/7455; A61B 5/7475; A61M 21/02; G16H 40/63; A61B 2562/0204; A61B 2562/0247; A61M 2021/0022; A61M 2021/0027; A61M 2021/005; A61M 2205/3303; A61M 2205/3327; A61M 2205/3331; A61M 2205/52; A61M 2230/005

(57) ABSTRACT

An apparatus for one or more of measuring cognitive state of a user and inducing a change of cognitive state of a user is disclosed. The apparatus is configured to: receive a response signal indicative of an active user response provided in response to a stimulus provided by a stimulus means; determine a control signal in dependence on a characteristic of the response signal and in accordance with a dynamic stimuli pattern, wherein the dynamic stimuli pattern is indicative of possible stimuli to be provided in response to the active user response; and provide the control signal to the stimulus means to control the stimulus means to provide a further stimulus in accordance with the control signal.

### TECHNICAL FIELD

The present invention relates to measuring a cognitive state of a user and inducing a change of cognitive state of a user, particularly, by accounting for the user's active response to stimuli using what may be termed “Active-Feedback”. Aspects relate to an apparatus, a system, a computer-implemented method and computer software.

### BACKGROUND

Understanding cognitive function of a user (who may also be called a subject, e.g. a human), is important in many clinical and behavioural settings.

For example, sleep disorders, including difficulties in either falling asleep or remaining asleep, are increasingly common; one in three adults in the UK are reported to suffer from a sleep disorder of some kind. A lack of sleep can result in impaired concentration and lengthened reaction times whilst awake, as well as a general feeling of tiredness. Difficulties in falling asleep can be caused by an inability to ignore conscious thoughts, which may be caused by stress or anxiety. Other sleep issues may be that a person struggles to maintain a sleep cycle at an appropriate time of day, for example due to jet lag or that person's personal circadian rhythm being out of sync with a desired sleep pattern.

Understanding a person's cognitive function in relation to cognitive conditions such as Alzheimer's disease and other forms of dementia is also important, to understand any progression of the condition and guide possible treatments and activities to aid the person in reducing a negative impact on their quality of life.

Regarding sleep, to aid falling asleep, some approaches include meditative activity in which a stimulus is provided to encourage the user to relax, for example by providing a light whose intensity varies cyclically so that the user may match the frequency of their breathing to frequency of the cycles of the light. In this way the frequency of the cycles of the light may be gradually reduced so that the user is encouraged to slow their breathing at a predetermined rate. While this provides an activity with limited side effects, this approach may be considered to be passive, because there is no engagement with the user or any adaptation to what the user is doing. Thus they may be ineffective for certain individuals or on occasions when the user finds it particularly difficult to get to sleep.

In the context of dementia and other cognitive function disorders, it is important to be able to monitor a persons cognitive abilities. If possible, improving a person's cognitive ability (and this may mean maintaining current cognitive abilities, increasing cognitive abilities, or slowing down a decline in cognitive ability) is desirable in many cases. It is challenging to be able to provide activities which can both monitor a person's cognitive functional ability as well as aim to improve it, especially where the person may suffer from emotional challenges such as frustration or confusion.

It is an object of embodiments of the invention to at least mitigate one or more of the problems of the prior art.

### SUMMARY

In an aspect, there is provided an apparatus for one or more of measuring cognitive state of a user and inducing a change of cognitive state of a user, the apparatus configured to: receive a response signal indicative of an active user response provided in response to a stimulus provided by a stimulus means; determine a control signal in dependence on a characteristic of the response signal and in accordance with a dynamic stimuli pattern, wherein the dynamic stimuli pattern is indicative of possible stimuli to be provided in response to the active user response; and provide the control signal to the stimulus means to control the stimulus means to provide a further stimulus in accordance with the control signal.

The apparatus may be further configured to: receive a further response signal indicative of a further active user response provided in response to the further stimulus; and determine a further control signal in dependence on a characteristic of the first and further response signals and in accordance with the dynamic stimuli pattern.

The characteristic of the response signal may comprise one or more of:
a time delay between the provision of the stimulus and the receipt of the response signal in response to the stimulus;
a duration of provision of the active user response;
a pressure applied to provide the active user response;
a speed of provision of the active user response;
a determined user position during provision of the active user response;
a volume of the active user response when the active user response is a sound; and
an accuracy of response provision.

The dynamic stimuli pattern may comprise a reduction in one or more of stimulus amplitude and stimulus frequency as a function of time, wherein the rate of the reduction of the one or more of stimulus amplitude and stimulus frequency is dependent on the characteristics of the response signal.

The dynamic stimuli pattern may comprise an increase in one or more of stimulus amplitude and stimulus frequency as a function of time, wherein the rate of the increase of the one or more of stimulus amplitude and stimulus frequency is dependent on the characteristics of the response signal.

The rate of the one or more of stimulus amplitude and stimulus frequency may be further dependent on a passive user status signal received from a biosensor. The dynamic stimuli pattern 300 may be determined by using machine learning in some examples, for example using a passive user status signal to predict how a user is likely to respond to particular stimuli and determining the dynamic stimuli pattern 300 accordingly.

The dynamic stimuli pattern may comprise successive stimuli having one or more of substantially the same stimulus amplitude and stimulus frequency.

The dynamic stimuli pattern may comprise a plurality of stimulus periods, each of the stimulus periods being indicative of stimuli within a predetermined stimulus parameter range, and the predetermined stimulus parameter range of each stimulus period being at least partially different from the predetermined stimulus parameter range of each other stimulus period. The control signal may be determined according to a subsequent stimulus period once an advance criterion of a current stimulus period is met as determined according to the characteristic of the response signal. That is, the plurality of stimuli periods may each have an associated variable time duration; and the time duration of each of the stimuli periods may be determined according to the advance criterion of a current stimulus period being met as determined according to the characteristic of the response signal.

The dynamic stimuli pattern may further comprise a rest period between consecutive stimulus periods, and wherein, during the rest period, no stimuli are provided. The length of time of the rest period may be longer between later stimuli periods than early stimuli periods.

The predetermined stimulus parameter range may indicate one or more of:
light stimuli within a predetermined amplitude range;
light stimuli within a predetermined wavelength range;
light stimuli within a predetermined frequency range;
light stimuli within a predetermined lumen range;
haptic stimuli within a predetermined amplitude range;
haptic stimuli within a predetermined duration range;
haptic stimuli within a predetermined duty range;
audio stimuli within a predetermined volume range;
audio stimuli within a predetermined pitch range;
audio stimuli within a predetermined duration range; and
a number of stimuli concurrently output as a stimulus group.

The advance criterion of a current stimulus period may comprise one or more of: an active user response reaction time being outside a predetermined reaction time period; a number of active user responses being absent following a stimulus; when the active user response is provided via application of pressure to a pressure sensor, an active user response having a pressure below a predetermined pressure threshold; when the active user response is provided via provision of audio to an audio sensor, an active user response having a volume below a predetermined volume threshold; and when an incorrect active user response is provided in response to a cognitive task stimulus.

The advance criterion of a current stimulus period may comprise one or more of: an active user response reaction time being within a predetermined reaction time period; a threshold number of active user responses being made following respective stimuli; when the active user response is provided via application of pressure to a pressure sensor, an active user response having a pressure above a predetermined pressure threshold; when the active user response is provided via provision of audio to an audio sensor, an active user response having a volume above a predetermined volume threshold; and when an correct active user response is provided in response to a cognitive task stimulus.

The control signal may be further determined according to a subsequent stimulus period once a passive user advance criterion is met as determined according to a passive user status signal received from a biosensor. For example, the biosensor may be a heart rate sensor, electrodermal activity sensor, electromyography sensor, respiration sensor, perspiration sensor, skin temperature sensor or blood pressure sensor.

The control signal may be further determined according to a subsequent stimulus period when a duration for which a current stimulus period is in use reaches a current stimulus period threshold.

The dynamic stimuli pattern may be indicative of a plurality of possible stimuli within a predetermined amplitude range; and one or more spike stimuli having an amplitude outside the predetermined amplitude range. The apparatus may be configured to: determine the control signal in accordance with the one or more spike stimuli if the characteristic of the response signal is indicative of an active user response outside a predetermined response range.

The apparatus may be configured to: determine that the user is asleep in dependence on a characteristic of one or more response signals; and stop the provision of stimuli in dependence on determining that the user is asleep.

The apparatus may be configured to stop the provision of stimuli once a predetermined session time period for providing stimuli is reached.

The apparatus may be configured to: determine that the user is asleep in dependence on a characteristic of one or more response signals; pause the dynamic stimuli pattern to pause the provision of stimuli in dependence on determining that the user is asleep; determine that the user is awake in dependence on receipt of a passive user status signal received from a biosensor; if the user is determined to re-awaken within a predetermined period of time following the pause in provision of stimuli, recommence the provision of stimuli according to the paused dynamic stimuli pattern; and if the user is determined to re-awaken after a predetermined period of time following the pause in provision of stimuli, recommence the provision of stimuli according to the start of a dynamic stimuli pattern.

The apparatus may be configured to store the characteristics of the response signals provided by the user in response to the stimuli in a user response log. The user response log may be stored at a remote server and the apparatus may be configured to communicate with the remote server to store the characteristics of the response signal in the user response log and retrieve data from the user response log. The dynamic stimuli pattern may be determined in accordance with the response log. The dynamic stimuli pattern may be determined by using machine learning in some examples.

The apparatus may be configured to: receive a sleep pattern change signal indicative of a requested change in sleep pattern; and determine the dynamic stimuli pattern in accordance with a recorded current sleep time indicative of a time at which a user currently goes to sleep, and the requested change in sleep pattern, to induce sleep at a different time to the current sleep time.

The apparatus may be configured to determine the dynamic stimuli pattern according to a current time of day. For example, if the intention is to go to sleep starting at night-time, then less intense stimuli and/or increased reaction times may be used, whereas if the intention is to go to sleep in the middle of the day, then more intense stimuli and/or shorter reaction times may be used.

The apparatus may be configured to further provide an ambient control signal to an ambient stimulus apparatus in accordance with a stored user profile. The ambient stimulus apparatus may be a lamp, an audio output device, or a thermostat controlled heating or cooling device, for example. The apparatus may be configured to determine the ambient control signal further in accordance with the stimuli.

The apparatus may be a processor, a portable electronic device, a mobile telephone, a docking station, or a server.

In another aspect there is provided a system for one or more of measuring cognitive state of a user and inducing a change of cognitive state of a user, the system comprising any apparatus discloses herein, and stimulus means. The system may further comprise a response means configured to receive the active user response. The stimulus means may comprises one or more of: a light output device; an image output device; an audio output device; and a haptic output device.

In another aspect there is provided a computer-implemented method for one or more of measuring cognitive state of a user and inducing a change of cognitive state of a user, the method comprising: receiving an response signal indicative of an active user response provided in response to a stimulus provided by a stimulus means; determining a control signal in dependence on a characteristic of the response signal and in accordance with a dynamic stimuli pattern, wherein the dynamic stimuli pattern is indicative of possible stimuli to be provided in response to the active user response; and providing the control signal to the stimulus means to control the stimulus means to provide a further stimulus in accordance with the control signal.

The method may further comprising: providing the stimulus by the stimulus means; and providing the further stimulus by the stimulus means in accordance with the control signal.

In another aspect there is provided computer software which, when executed on a processor of any apparatus disclosed herein, or any system disclosed herein, is arranged to perform any method disclosed herein.

In another aspect there is provided a non-transitory, computer-readable storage medium storing instructions thereon that, when executed by one or more electronic processors of any apparatus disclosed herein, or of any system disclosed herein, causes the one or more electronic processors to carry out any method disclosed herein.

The term “computer” may be understood to encompass a single computing entity/device or a distributed computer system comprising a plurality of computing entities/devices which may be located at substantially the same geographic location, or at substantially different geographical locations. One or more computing entities/devices in a distributed system may be located in the “computing cloud”.

### BRIEF DESCRIPTION OF THE DRAWINGS

Embodiments will now be described by way of example only, with reference to the accompanying figures, in which:

FIG. 1 shows an apparatus according to examples disclosed herein;

FIG. 2 shows an apparatus and stimulus means according to examples disclosed herein;

FIGS. 3a-3b show examples of a dynamic stimuli patterns according to examples disclosed herein;

FIG. 4 shows an example of providing stimuli in relation to a user sleeping according to examples disclosed herein

FIG. 5 shows a system according to examples disclosed herein;

FIG. 6a shows an example of stimuli provided over a time period before any adaptation in response to provided user responses according to examples disclosed herein;

FIG. 6b shows an example of stimuli provided over a time period including adaptation of the stimuli provided in response to provided user responses according to examples disclosed herein; and

FIG. 7 shows a method according to examples disclosed herein.

### DETAILED DESCRIPTION

Examples disclosed herein relate to apparatuses, methods and computer programs which involve what may be termed “Active-Feedback”. Active-Feedback may be understood to mean that a response is consciously provided by a user in response to a stimulus, in order to generate a subsequent stimulus which is based at least partly on the previously provided response. Some examples disclosed herein may use such Active-Feedback methods to measure a cognitive state of a user; for example, the nature of the user's responses in response to provided stimuli may be used to determine a characteristic of the user's cognitive state. Some examples disclosed herein may use such Active-Feedback methods to induce a change of cognitive state of a user; for example, the nature of the user's response in response to the provided stimuli may control the provision of subsequent stimuli to induce a particular cognitive state, for example, relaxation, sleeping, mental stimulation, or waking. By using Active-Feedback to take account of a user's conscious response to stimuli, to provide subsequent stimuli, the level of engagement of the user may be accounted for which may provide improved results from the provision of stimuli to the user compared to a passive system by which no account of the user's active engagement is made.

Systems where a user is subjected to stimuli without real-time feedback from the user to control the stimuli, such as a lamp operating according to a pre-programmed illumination program, maybe considered to be passive systems, because there is no engagement with the user or any adaptation to what the user is doing. By using a user's responses to stimuli to determine subsequent stimuli as in examples disclosed herein, a user's current state can be accounted for and used to tailor the stimuli provision according to the particular user's current state.

Systems disclosed herein which use Active-Feedback to account for the user's current cognitive state in providing subsequent stimuli, may be used in various scenarios. For example, providing stimuli to a user to induce sleep, or induce waking, may be achieved effectively because the current behaviour of the user is used, by way of the provided responses from the user, to control the nature of subsequent stimuli. In the context of monitoring a person's cognition suffering from dementia or other cognitive function disorders, accounting for a user's cognitive state in real time during stimuli provision may allow for improvement in the monitoring of person's cognitive abilities. In some examples, a tailored stimuli program may be provided according to the person's current behaviour, as determined by their responses, to try and maintain, improve, or slowing down a decline in their cognitive ability.

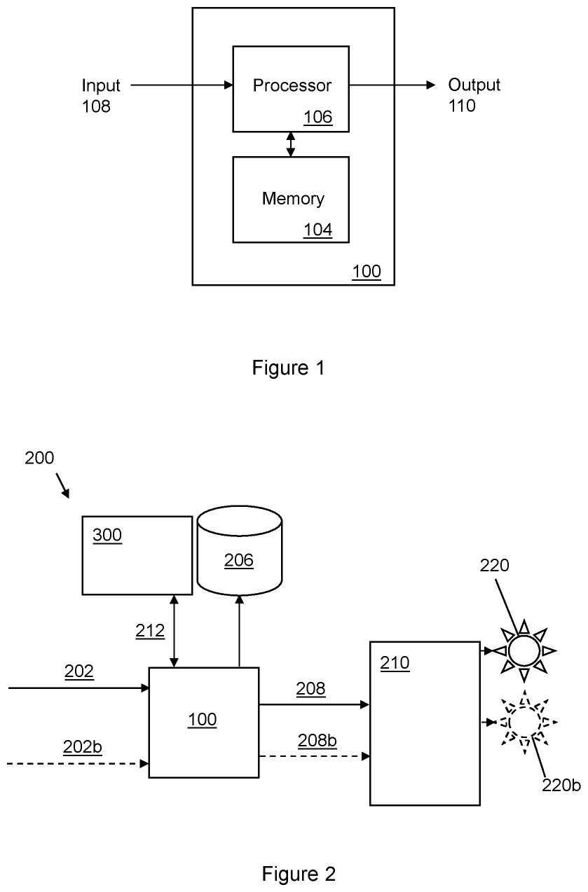

FIG. 1 shows an example apparatus 100 comprising a memory 104 and processor 106. The apparatus 100 in some examples may comprise one or more processors 106 (only one processor 106 is shown in this example). The processor(s) 106 may be arranged to operably execute computer software/computer program code thereon, where the computer software/computer program code is stored in a computer-readable medium accessible to the processor(s) 106. The computer-readable medium may be a memory device or memory 104 of the apparatus 100. Data for use by the software/program code may also be stored in a memory (e.g. the apparatus memory 104 or a separate memory store external to the apparatus 100). The memory 104 in some examples may be part of a physically distinct computer system than the one or more computers implementing the processing, and may in some examples be located at a remote server or cloud. The processor(s) 106 do not necessarily need to be formed as part of the same apparatus 100 and at least a portion of plural processors 106 may form a virtual machine e.g. as a cloud computer. Some examples may be implemented by a plurality of distributed apparatus 100, with each of the apparatus 100 performing one or more processing steps.

The apparatus 100 can receive data as input 108 (e.g. a response signal 108 indicative of an active user response provided in response to a stimulus provided by a stimulus means). The apparatus 100 can process data at the processor 106 (e.g. the processor can process the received active user response signal and determine a control signal in dependence on a characteristic of the response signal and in accordance with a dynamic stimuli pattern as discussed below). The apparatus 100 can provide data as output 110 (e.g. a control signal 110 may be provided to the stimulus means, to control the stimulus means to provide a further stimulus in accordance with the control signal). An active user response may be considered to be an input which is consciously, or deliberately, input or provided by a user in response to a provided stimulus which would not otherwise have been made. That is, the user makes a choice to react to a stimulus, as opposed to any response signal which is passive and provide unconsciously by the user. The active response may be made, for example, by pressing or tapping a button, selecting an option of plural presented options, making a sound or speaking a word, squeezing or applying pressure to a pressure sensitive input device, or other consciously-made input.

FIG. 2 shows an example process 200 which can be performed in an apparatus 100. The apparatus 100 may be used to measure the cognitive state of a user in some examples. The apparatus 100 may be used to induce a change of cognitive state of a user in some examples. The apparatus 100 is configured to receive a response signal 202 which is indicative of an active user response provided in response to a stimulus 220 provided by a stimulus means 210. For example, the stimulus may be a light illuminating, and the user may make an input (e.g. press a button, make a sound, tap an icon) in response to seeing the light which causes a response signal due to the user's input to be provided to the apparatus 100.

The apparatus 100 is configured to determine a control signal 208 in dependence on a characteristic of the response signal 202 and in accordance with a dynamic stimuli pattern 300. For example, a characteristic of the response signal 202 may be the time difference between the stimulus being provided and the time of the active user input being made, or may be the pressure with which the active user input is made (e.g. to a button or other pressure-sensitive device). The characteristic of the response signal may more generally comprise one or more of: a time delay between the provision of the stimulus and the receipt of the response signal in response to the stimulus; a duration of provision of the active user response; a pressure applied to provide the active user response; a speed of provision of the active user response; a determined user position during provision of the active user response; a volume of the active user response when the active user response is a sound; and an accuracy of response provision (for example, if the stimulus is an object displayed on a touch screen, the accuracy may be the distance away from the object that the user touches the touch screen).

The dynamic stimuli pattern 300 is indicative of possible stimuli to be provided in response to the active user response 202. For example, it may define a range of possible light stimuli within respective colour/frequency, intensity, or illumination duration ranges, or it may define a plurality of possible images, one of which can be selected according to the user's active response and provided for display as a subsequent stimulus. The dynamic stimuli pattern 300 is discussed in more detail below.

The apparatus 100 is configured to provide the control signal 208 to the stimulus means 210, to control the stimulus means 210 to provide a further stimulus 220b in accordance with the control signal 208. Therefore, the response of the user is accounted for when providing a subsequent stimulus 220b which is also in accordance with possible permitted stimuli as defined in the dynamic stimuli pattern 300. The dynamic stimuli pattern 300 may be considered to be “dynamic” in that a subsequent stimulus 220b defined in the dynamic stimuli pattern 300 is dynamically selected from possible options in the pattern in response to the response provided by the user-a slower response may cause a different stimulus to be provided than a faster response, for example. The dynamic stimuli pattern 300 may be considered to be a “pattern” in that it pre-defines a series of stimuli which follow a predetermined trend, which may be tailored to a particular usage. For example, if the apparatus 100 is used in a scenario to help a user relax or go to sleep, the pattern 300 may include generally decreasing stimulus intensity as the user gradually relaxes. As another example, if the apparatus 100 is used in a scenario to understand a user's cognitive functioning relating to recognition of images, the pattern 300 may include generally more obscured or difficult to recognise images as the user progresses through the pattern 300.

The apparatus 100 may be further configured to receive a further response signal 202b indicative of a further active user response provided in response to the further stimulus 220b, and determine a further control signal 208b in dependence on a characteristic of the first and further response signals 220, 220b and in accordance with the dynamic stimuli pattern 300. For example, once the user has made a first response 202 to a first stimulus provided by the stimulus means 210, the first response 202 determines, in part, a further stimulus 220b provided by the stimulus means 210, which the user also responds to in a further response 202b.

The apparatus 100 may be configured to store the characteristics of the response signals provided by the user in response to the stimuli in a user response log 206. In some examples, the user response log 206 may be stored at a remote server and the apparatus 100 may be configured to communicate with the remote server to store the characteristics of the response signal 202, 202b in the user response log 206 and retrieve data from the user response log. In other examples, the user response log 206 may be stored on a memory of a device which also comprises the apparatus 100. The dynamic stimuli pattern 300 may be determined in accordance with the response log 206. For example, the previously logged response behaviour of the user may be used to generate a user-specific dynamic stimuli pattern 300. As an example, a user may wish to increase their alertness or focus and may respond well to a dynamic stimuli pattern providing haptic stimuli. The same person may wish to be induced into a sleeping state at 11 pm, and may respond poorly to haptic stimuli but respond well to light stimuli. This feedback system advantageously provides stimuli which are suited to the user's personal habits and biological sleeping/waking traits.

The dynamic stimuli pattern 300 may be determined by using machine learning in some examples, for example using the user's previous response inputs as test data (and in some examples, test data from one or more other user's, for example of a similar demographic and the user) to adapt the dynamic stimuli patterns provided to improve effectiveness for the user.

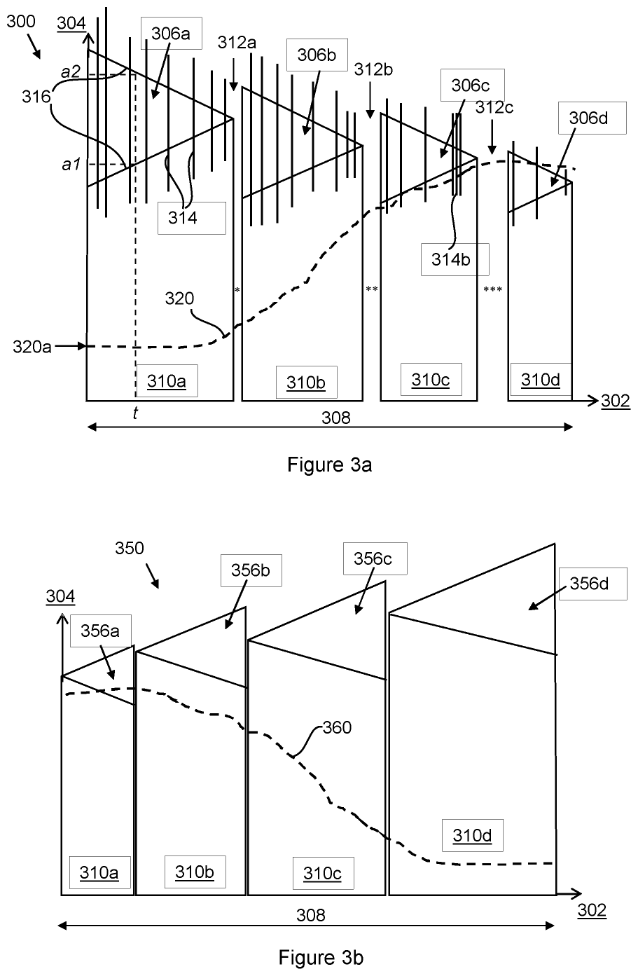

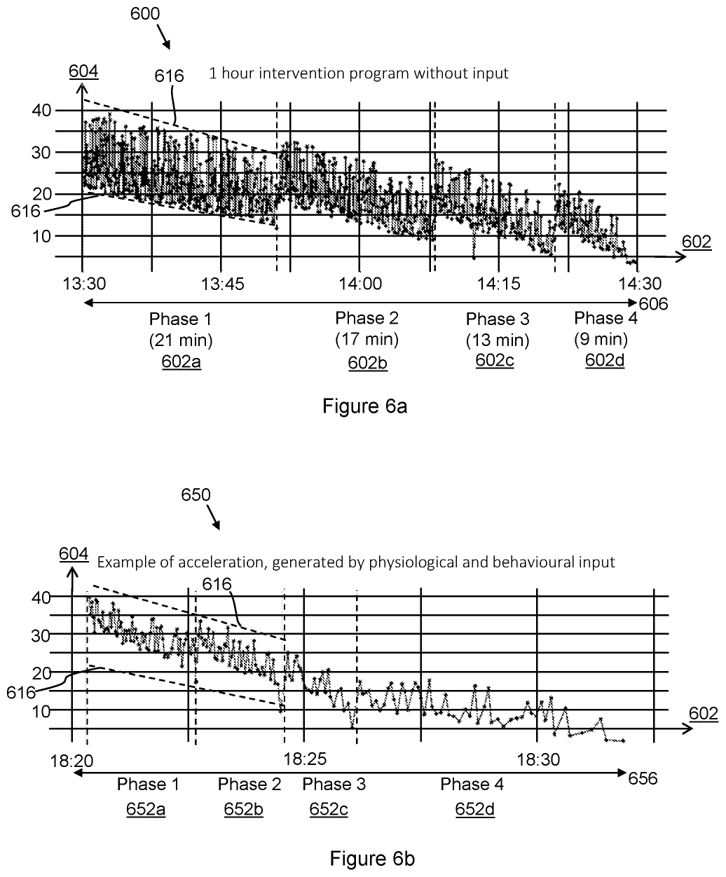

FIGS. 3a and 3b show schematic examples of a dynamic stimuli pattern 300, 350. Note that example data sets shown in FIGS. 6a and 6b are examples of the schematic dynamic stimuli pattern of FIG. 3a. A range of possible stimuli amplitudes 304 are indicated by the triangular regions 306a-d, 356a-d at different points in time 302 over a period of time 308. The dynamic stimuli patterns 300, 350 are indicative of possible stimuli to be provided in response to an active user response.

In the example of FIG. 3a, the dynamic stimuli pattern 300 comprises a reduction in stimulus amplitude 304 as a function of time 302. In some examples the dynamic stimuli pattern 300 may comprise a reduction in stimulus frequency as a function of time (as well, or instead of, a reduction in stimulus amplitude). That is, over the period of time 308 of stimulus provision, the stimulus amplitude 304 of possible stimuli generally decreases. In this example, where the possible stimuli are separated into discrete periods 310a-d (which may also be called sub-periods, phases, blocks, or sections for example), the stimulus amplitude 304 of possible stimuli may generally decrease over each period 310a-d as well.

In this example, the dynamic stimuli pattern 300 comprises a plurality of stimulus periods 310a-d. Each of the stimulus periods is indicative of stimuli within a predetermined stimulus parameter range 306a-d. The predetermined stimulus parameter range 306a-d of each stimulus period 310a-d may be, at least partially, different from the predetermined stimulus parameter range 306a-d of each other stimulus period 310a-d, as shows by the triangular regions 306a-d each covering at least partially different ranges of stimuli amplitude 304. The stimuli output amplitude and/or frequency in this example reduces on average over the whole duration 308, and also the stimuli output amplitude and/or frequency reduces on average within each of the periods 310a-d. The start of each period 310a-d can include stimuli of amplitude and/or frequency which is higher than the stimuli amplitude and/or frequency at the end of the previous period 310a-d. The start of each period 310a-d may not include stimuli of amplitude and/or frequency which is higher than the stimuli amplitude and/or frequency at the start of the previous period 310a-d. These characteristics of the amplitude and/or frequency ranges of adjacent periods 310a-d, resulting in an increase at the start of a new period 310a-d, may serve to increase/grab the user's attention, and allow the next stimuli period to be performed as a new series of less intense/frequent stimuli.

The rate of the reduction of the stimulus amplitude and/or stimulus frequency may be dependent on the characteristics of the response signal. For example, the example stimulus pattern 300 may be used to induce a sleeping state of the user. If a user is providing very rapid responses, this may be taken as an indication that the user is very awake and alert, and so the rate of reduction of the stimuli amplitudes and/or frequency may be low until the user starts to show an indication of becoming more relaxed (e.g. response times to the provided stimuli are longer). Conversely, if a user is providing very slow or very light (button-press) responses, this may be taken as an indication that the user is tired and falling asleep, and so the rate of reduction of the stimuli may be high, because the user appears to be almost asleep and the provision of stimuli may soon not be required so the higher rate of reduction is applied to conclude the provision of stimuli.

In examples where the stimuli provided are light stimuli (e.g. a light or series of lights), the predetermined stimulus parameter range 306a-d may indicate one or more of light stimuli within a predetermined amplitude range (e.g. brightness); light stimuli within a predetermined wavelength range (e.g. colour or colours); light stimuli within a predetermined frequency range (e.g. colour or colours); and light stimuli within a predetermined lumen range (e.g. the amount of light emitted). In examples where the stimuli provided are haptic or touch stimuli such as a vibrator or tactile mat/pad providing touch stimuli, the predetermined stimulus parameter range 306a-d may indicate one or more of haptic stimuli within a predetermined amplitude range (e.g. intensity of vibration, size of tactile cue); haptic stimuli within a predetermined duration range (e.g. length of vibration); and haptic stimuli within a predetermined duty range (e.g. the time taken for a stimulus to be provided then removed, repeated). In examples where the stimuli provided are audio stimuli, the predetermined stimulus parameter range 306a-d may indicate one or more of audio stimuli within a predetermined volume range; audio stimuli within a predetermined pitch range; and audio stimuli within a predetermined duration range. In examples where the stimuli provided are a plurality of concurrently provided stimuli (e.g. plural displayed visual cues, plural sounded audio cues, or plural cues of different types e.g. light and sound), the predetermined stimulus parameter range 306a-d may indicate a number of stimuli concurrently output as a stimulus group.

The dynamic stimuli pattern 300 of FIG. 3a also comprises rest periods 312a-c between consecutive stimulus periods 310a-d. During the rest period 312a-c, no stimuli are provided. The length of time of the rest period 312a-c may be longer between later stimuli periods than earlier stimuli periods in some examples, such as the example of FIG. 3a where the overall stimuli amplitude and/or frequency decreases generally as the dynamic stimuli pattern 300 progresses with time. The length of time of the rest period 312a-c may be shorter between later stimuli periods than earlier stimuli periods in some examples, such as an example where the overall stimuli amplitude and/or frequency increases generally as the dynamic stimuli pattern 300 progresses with time.

A dynamic stimuli pattern 300, as shown in FIG. 3a, may be indicative of a plurality of possible stimuli within a predetermined amplitude range 316 and one or more spike stimuli 314 having an amplitude outside the predetermined amplitude range 316. The predetermined amplitude range 316 of possible stimuli is illustrated as being bound by the sloping lines forming edges of the triangle 306a which indicates the area within which the amplitude of possible stimuli can be selected at a particular time. For example, at time t, stimuli within the amplitude range of a1 and a2 may be provided as possible stimuli within the predetermined amplitude range 316. Spike stimuli 314 may be provided periodically or intermittently with varying time periods between them, and may help to maintain a user's attention by providing a stimulus outside the usual stimuli amplitude parameters and help avoid the user's attention drifting away. Such spike stimuli 314 may decrease, or increase, or be constant, in frequency as a function of time, at least within a stimulus period 310a-d. In some examples, such spike stimuli may decrease, increase, or have substantially constant amplitude as a function of time, at least within a stimulus period 310a-d. Earlier stimulus periods 310a-d may include more spike stimuli than later stimulus blocks 310 a-d in some examples (e.g. where an aim is for the user to relax). Earlier stimulus periods 310a-d may include fewer spike stimuli than later stimulus blocks 310a-d in some examples (e.g. where an aim is for the user to increase their focus or alertness). In some examples, the spike stimuli 314b may be provided in a cluster or burst of higher than expected amplitude and frequency stimuli as shown in the third period 310c in this example, but may appear in other periods and may appear more than once.

In some examples, the apparatus 100 may be configured to determine the control signal in accordance with the one or more spike stimuli 314 if the characteristic of the response signal is indicative of an active user response outside a predetermined response range. For example, if the user is responding more slowly that required for the current phase of the dynamic stimuli pattern 300 this may be an indicator that the user is not focused on the task of responding to the provided stimuli and the spike stimulus 314 may be provided to attract the user's attention back on the task of responding to subsequent stimuli. That is, to help mitigate against the user feeling that responding to the stimuli is boring (which may undesirably cause the user's attention to drift away from engaging in the tasks of responding), the possible spikes 314 on top of the underlying dynamic stimuli pattern 300 may be included. A spike may be considered to be a stimulus or group of stimuli of higher intensity, or stimuli provided in a burst of increased frequency.

In other examples, the predetermined amplitude range 316 of possible stimuli need not necessarily be a triangle space of possible stimuli but may, for example be defined as a rectangle, trapezium, semicircle, or other regular or irregular shape. The range of possible stimuli may not necessarily decrease with time for a given stimuli period in other examples.

In the specific example of the apparatus 100 being used to aid a person going to sleep as a change of cognitive state, the apparatus 100 may be configured to determine that the user is asleep in dependence on a characteristic of one or more response signals, and stop the provision of stimuli in dependence on determining that the user is asleep. For example, if the user does not provide a response signal for a predetermined “no response” period (e.g. 1 minute), and possibly if one or more passive indications match a sleep criterion (e.g. the user's skin temperature is within an expected sleeping temperature range, and/or the user's breathing rate is slower than the user's usual (previously determined and stored) awake breathing rate by a predetermined amount, then the user may be determined to be asleep and the apparatus can stop the provision of stimuli before the end of the period 308.

So, as an example, input from the user may be captured by a response device and provided to the apparatus 100 for processing as above and determination of a suitable output to control subsequent stimuli. The determination of the next stimulus to be provided may be made by the apparatus periodically (e.g. every second) in some examples. The combination of using a dynamic stimuli pattern 300, 350, which determines what stimuli to provide to measure or induce a change in a particular cognitive state, as well as accounting for the user's active responses, allows for suitable output to be provided which adapts to the user's determined cognitive state (as obtained from the user responses) but also which progresses to a desired cognitive state (e.g. sleep, alertness) irrespective of whether the user is actually progressing to that state. For example, if the goal is to induce sleep, providing stimuli according to the dynamic stimuli pattern 300, 350 provides for inducing sleep in the user irrespective of whether the user is actually growing sleepier. Thus the provided stimuli are not a direct reflection of either a preset program or a direct response to the user response, but a blend of at least those two factors.

Therefore, a dynamic stimuli pattern 300, 350 is selected for use which has a duration 308 of e.g. 30 minutes, then the provision of stimuli according to this pattern may last for 30 minutes if the is not physiologically or behaviourally changing their cognitive state (e.g. winding-down) which may accelerate the program. If the user does change their cognitive state (to wind down and become sleepy), their responses will be taken into account and used to provide a series of stimuli with accelerated characteristics for a user becoming sleepy (e.g. the amplitude of the stimuli reduces more quickly than if no indication of winding down of the user is determined). Thus the overall duration of the accelerated dynamic stimuli pattern 300, 350 may last for 20 minutes rather than 30 minutes. In other words, the stimuli may reduce in amplitude over time, irrespective of the responses provided by the user, but the speed by which this reduction in amplitude is made may be determined in real-time (e.g. every second) in dependence on the user's response inputs (indicative of their cognitive state).

In some examples, the dynamic stimuli pattern 300, 350 may be linked to fixed S-shaped curve describing an expected change in a particular physiology or behaviour characteristic (e.g. skin temperature, response time, breathing rate, pulse rate). These curves may be different for each characteristic, so for example the user's skin temperature (which may be detected as a passive response signal) may follow a different S-shaped curve than the user's reaction times (detected as an active response signal) and to the user's duration of missed responses to stimuli compared to responses made to stimuli. In some examples, the shape of each pre-defined curve for a characteristic may be adjusted, and may in some examples be adjusted to account for a user's personal historical physiological and behavioural traits. This may provide, for example, a S-shaped curve which is an average of the historical data for that user.

Real-time data may be checked against the S-shaped curve or curves for that user, allowing for an accurate level of acceleration suited to that user. This may be performed for one, more than one, or all the parameters sensed for the user by comparing each sensed parameter to the corresponding S-shaped curve for that parameter for the user, to determine whether to accelerate the programme of stimulus provision. In an example, each S-shaped curve for a particular parameter (e.g. breathing rate, response time) may be plotted as a multiplier factor between 0 and 1 on the y-axis against a range of possible parameter values on the x-axis.

For a given parameter value, there is therefore a particular multiplier factor. A stimulus period 310a-d may be accelerated through by periodically (e.g. every one second) checking the sensed/received value of the parameter against the S-shaped curve, and if there is a non-zero multiplier factor, then an acceleration of the current period may be made. For example, the time remaining in a stimulus period TRem may equal the original unmodified time remaining TOrig, minus the unmodified time taken to move through the stimulus period tOrig (e.g. one second), minus the time step taken to move through the stimulus period tOrig multiplied by the multiplier factor m (that is, TRem=TOrig−tOrig−(tOrig×m)). If the parameter does not indicate that there should be any acceleration (i.e. to reflect that the user is responding well to the stimuli) then m=0, and TRem =TOrig−tOrig, and so the program continues without acceleration and the time remaining counter reduced by one second (tOrig=1 s). If the parameter does indicate that there should be acceleration (e.g. a multiplier of 2 corresponds to the parameter value being considered) then TRem=TOrig−tOrig−2 tOrig, and so the program continues at an acceleration of 3× the unmodified rate of progression and the time remaining counter reduces by three seconds. The calculation may then be performed again in a loop to provide continuous (every one second) adjustment of the acceleration and time remaining in a stimulus period. For a tOrig (time step) of one second, this or similar calculation may be performed every one second to provide real-time acceleration reflecting the user's real-time state

If more than one parameter is considered, then each parameter may be assigned a weighting value. So, if breathing rate and response time are two considered parameters and breathing rate is considered to be half as important in determining an acceleration than response time, then any acceleration due to breathing rate may be given a weighting of 33% of the overall acceleration, and any acceleration due to response time may be given a weighting of 67% of the overall acceleration. Of course may other combinations of one or more parameters with appropriate weightings may be used in other examples.

In the example of FIG. 3b, the dynamic stimuli pattern 350 comprises an increase in stimulus amplitude 304 as a function of time 302 rather than the decrease shown in FIG. 3a. Features in common with or which can be understood from the description of FIG. 3a are not described again in detail here. In some examples the dynamic stimuli pattern 350 may comprise an increase in stimulus frequency as a function of time (as well, or instead of, a reduction in stimulus amplitude). That is, over the period of time 308 of stimulus provision, the stimulus amplitude 304 of possible 356a-d generally increases. In this example, where the possible stimuli 356a-d are separated into discrete periods 310a-d, the stimulus amplitude 304 of possible stimuli 356a-d may generally increase over each period 310a-d as well.

The rate of the increase of the stimulus amplitude 304 and/or stimulus frequency may be dependent on the characteristics of the response signal. For example, the example dynamic stimuli pattern 350 of FIG. 3b may be used to induce a more alert or aroused cognitive state, perhaps for a user who is initially sleepy or drowsy. If a user is providing very rapid responses, this may be taken as an indication that the user is very awake and alert, and so the rate of increase of the stimuli may be high because the user appears to be awake and the provision of stimuli may soon not be required so the higher rate of increase is applied to conclude the provision of stimuli. Conversely, if a user is providing very slow or very light (button-press) responses, this may be taken as an indication that the user is tired and still sleepy, and so the rate of increase of the stimuli may be lower, until the user starts to show an indication of becoming more alert (e.g. response times to the provided stimuli are shorter).

The example of FIG. 3b does not include the rest periods 312a-c shown in relation to FIG. 3a but in other examples a similar dynamic stimuli pattern 350 may also include such rest periods. The example of FIG. 3b does not include the spike stimuli 314 shown in relation to FIG. 3am but in other examples a similar dynamic stimuli pattern 350 may also include such spike stimuli 314.

In both the examples of FIGS. 3a and 3b, and other examples, the control signal to control the stimulus means may be determined according to a subsequent stimulus period (e.g. the second period 310b) once an advance criterion of a current stimulus period (e.g. the first period 310a) is met as determined according to the characteristic of the response signal. That is, the plurality of stimuli periods 306a-d may each have an associated variable time duration; and the time duration of each of the stimuli periods 310a-d may be determined according to the advance criterion of a current stimulus period being met as determined according to the characteristic of the response signal. The time period of each stimulus period 310a-d may vary, for example between predetermined minimum and maximum start and end times or to provide a timer period within predetermined time period limits.

The advance criterion of a current stimulus period 306a-d may comprise an active user response reaction time being outside a predetermined reaction time period. For example, if a user's response time is below a particular response time threshold (e.g. longer than 0.5 s), this may be an indication that the user is ready to move onto the next stimulus period. As another example, the advance criterion of a current stimulus period 306a-d in relation to FIG. 3a may comprise a number of active user responses being absent following a stimulus. For example, if a threshold number of user responses are absent following provision of an associated stimulus, this may be taken as an indication that the user is ready to move onto a subsequent stimulus period (e.g. the user is falling asleep so a series of lower amplitude stimuli can be provided in the subsequent stimulus period).

The advance criterion of a current stimulus period 306a-d may, when the active user response is provided via application of pressure to a pressure sensor, comprise an active user response having a pressure below a predetermined pressure threshold. The advance criterion of a current stimulus period 306a-d may, when the active user response is provided via provision of audio to an audio sensor (e.g. the user speaks a response or otherwise makes a noise as a response), comprise an active user response having a volume below a predetermined volume threshold. For example, if a user is to press a button to provide a response, and the pressure detected as being applied to the button is below a predetermined pressure threshold, and/or if a user is to make a sound to provide a response, and the pressure detected as being applied to the button is below a predetermined pressure threshold and/or the volume of the provided sound is below a predetermined audio threshold, this may be taken as an indication that the user is more tired or more relaxed and the next stimuli to be provided can be from the next portion of the dynamic stimuli pattern.

The advance criterion of a current stimulus period 306a-d may comprise an incorrect active user response being provided in response to a cognitive task stimulus. For example, a user may be provided with a coloured light stimulus for a particular period of time (e.g. 1 s) and the user is to press a button when the light is illuminated and the light colour is red. If the user presses the button when the light is blue, for example, this is an incorrect response and may be an indication that the user is becoming tired, so the subsequent stimuli may be provided from a subsequent stimulus period (wherein, for example, the light is shown for a longer period e.g. 2 s).

The above examples of an advance criterion may apply to scenarios in which the user may be induced into a more relaxed, sleepy, or calm cognitive state. Conversely, in examples in which the may be induced into a more alert or focussed cognitive state, the advance criterion of a current stimulus period may comprise one or more of: an active user response reaction time being within a predetermined reaction time period; a threshold number of active user responses being made following respective stimuli; when the active user response is provided via application of pressure to a pressure sensor, an active user response having a pressure above a predetermined pressure threshold; when the active user response is provided via provision of audio to an audio sensor, an active user response having a volume above a predetermined volume threshold; and when a correct active user response is provided in response to a cognitive task stimulus.

In both the examples of FIGS. 3a and 3b, and other examples, the rate of the one or more of stimulus amplitude and stimulus frequency may be further dependent on a passive user status signal received from a biosensor. For example, the biosensor may be a heart rate sensor, electrodermal activity sensor, electromyography sensor, respiration sensor, perspiration sensor, skin temperature sensor or blood pressure sensor. For example, a user's breathing rate, heart rate, skin temperature, pulse rate, or other passive biomarker may be suitably sensed and used as a passive indication of the cognitive state of the user. A passive indication may be considered to be a sensed property of the user's cognitive state determined without the user consciously providing any input or response, i.e. it is an inherent indicator of the user's current cognitive state determined without deliberate user input.

The control signal may be further determined in some examples according to a subsequent stimulus period once a passive user advance criterion is met as determined according to a passive user status signal received from a biosensor. The schematic curves 320, 360 in FIGS. 3a and 3b illustrate the skin temperature of the user throughout the duration of stimuli provision according to the dynamic stimuli pattern 300, 350. For example, in relation to FIG. 3a and the discussion of acceleration above, if the user's skin temperature is determined to indicate that a multiplier factor is to be applied to the time step through the stimulus period, the remaining stimulus period may be accelerated and the next stimulus period started more quickly. For example, the programme may accelerate by a predetermined amount of time, e.g. 1 second, and if the user's body temperature indicates the user is responding well, such that a multiplier factor is to be applied, the program may accelerate by an accelerated amount of 1.2 seconds to more quickly reach the next stimulus period. Acceleration may be based on an active characteristic (e.g. response time, number of stimuli left unresponded-to), passive (e.g. pulse rate, skin temperature), or both types of characteristic. In examples where an acceleration is made through the programme of stimuli which may be provided by the dynamic stimuli pattern, the overall length of the pattern may remain the same as without any acceleration. That is, the total duration of the dynamic stimuli pattern need not be shortened and where an earlier portion is accelerated through (i.e. and lasts for a shorter time), because the time which is not spent in that earlier portion may be then spent in a later portion of the programme. That is, where one period may be used for a shorter period of time, another period may then be used for a longer period of time to compensate and maintain a constant dynamic stimuli pattern length of time.

The control signal may be further determined in some examples according to a subsequent stimulus period when a duration for which a current stimulus period is in use reaches a current stimulus period threshold. For example, a maximum duration for the first period 310a in FIG. 3a may be 12 minutes, and when stimuli according to the first stimulus period 310a have been provided for the maximum duration of 12 minutes, the next stimulus is made according to the next stimulus period 310b. The apparatus may be configured in some examples to stop the provision of stimuli once a predetermined session time period for providing stimuli is reached. Foer example, once the period of time 308 of stimulus provision has elapsed, no further stimuli may be provided in that session.

In other examples, the dynamic stimuli pattern may comprise successive stimuli having one or more of substantially the same stimulus amplitude and stimulus frequency. For example, if the provided stimuli are photographic images, these may be considered not to have a particular “amplitude” (or of some property of the image such as brightness is considered to be an amplitude, this property is substantially the same over plural stimuli). In such examples, the user may provide an active user response in response to, for example, recognising a family member in a displayed photograph or being able to say the name of a colour when that colour is displayed to the user within a predetermined period of time from the colour being displayed.

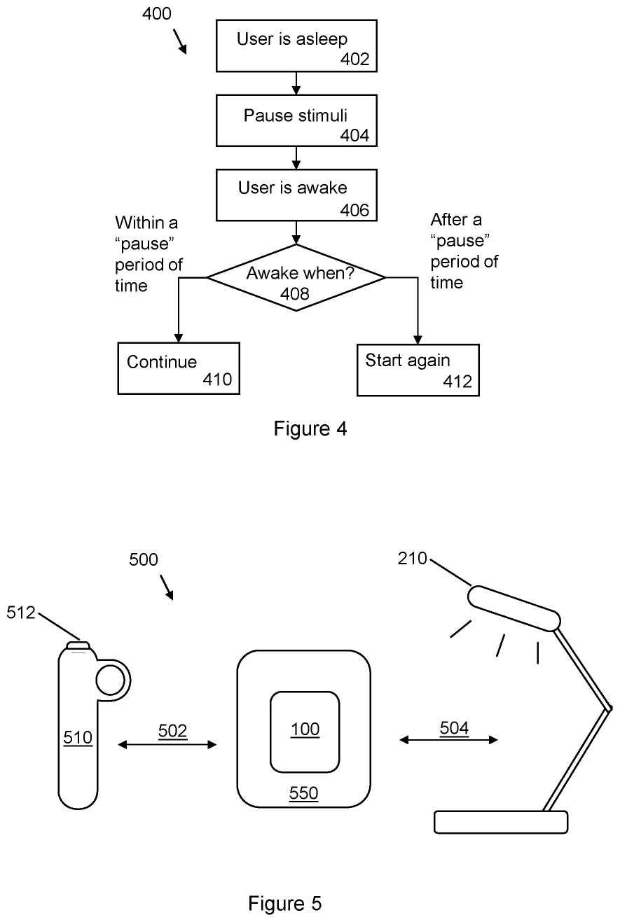

FIG. 4 shows an example process 400 taking place in the apparatus 100 specifically in relation to a user falling asleep. In some examples, the apparatus 100 may be configured to determine that the user is asleep 402 in dependence on a characteristic of one or more response signals. This may be that the user is providing a response outside a predetermine response period, for example. The apparatus 100 may then pause the dynamic stimuli pattern 404 to pause the provision of stimuli in dependence on determining that the user is asleep. The apparatus may then determine that the user is awake in dependence on receipt of a passive user status signal 406 received from a biosensor. For example, the user's breathing rate may quicken or the user's skin temperature may fall, for example. If the user is determined to re-awaken within a predetermined period of time following the pause in provision of stimuli, the apparatus 100 may be configured to recommence the provision of stimuli 410 according to the paused dynamic stimuli pattern. That is, the provision of stimuli may carry on from where it was paused previously, because the user has not been determined to have been sleeping for very long. If the user is determined to re-awaken after a predetermined period of time following the pause in provision of stimuli, then the apparatus 100 may be configured to recommence the provision of stimuli 412 according to the start of a dynamic stimuli pattern. That is, the user may be determined to have been asleep for a period of time which is long enough that they user does not need to receive any further stimuli from the paused dynamic stimuli pattern on awakening; they are likely to be awake and active until a later sleep session.

The apparatus in some examples may be configured to receive a sleep pattern change signal indicative of a requested change in sleep pattern; and determine the dynamic stimuli pattern in accordance with a recorded current sleep time indicative of a time at which a user currently goes to sleep, and the requested change in sleep pattern, to induce sleep at a different time to the current sleep time. For example, a user may wish to shift their sleep time of day due to changing time zone.

The apparatus may be configured to determine the dynamic stimuli pattern according to a current time of day. For example, if the intention is to go to sleep starting at night-time, then less intense stimuli and/or increased reaction times may be used, whereas if the intention is to go to sleep in the middle of the day, then more intense stimuli and/or shorter reaction times may be used to help counteract the effect of the daytime making it harder to fall asleep.

FIG. 5 shows an example system 500 according to some examples which is for one or more of measuring cognitive state of a user and inducing a change of cognitive state of a user. The system 500 comprises an apparatus 100 which in this example is part of a device 550 (e.g. a processor, a portable electronic device, a mobile telephone, a docking station, or a server) but in other examples the apparatus 100 itself may be considered to be a processor, a portable electronic device, a mobile telephone, a docking station, or a server. The system 500 also comprises stimulus means 210, such as a light output device (e.g. a lamp); an image output device (e.g. a display screen); an audio output device (e.g. a speaker); and/or a haptic output device (e.g. a vibrator), or other device configured to provide audio, visual and/or haptic stimuli to a user. The apparatus 100 and the stimulus means 210 may communicate 504 with each other. The system 500 may further comprise, as shown in FIG. 5, a response means 510 which is configured to receive the active user response. The apparatus 100 and the response means 210 may communicate 502 with each other. For example, the response means 510 may comprise a button 512, microphone, pressure sensor, or other input means by which the user can respond to a stimulus. In some examples, the response means may be a remote control.

In some examples, the stimulus means 210 and the response means 510 may be the same device, for example a handheld device which can vibrate or provide a sound as a stimulus, and which comprises one or more input means for the user to provide a response. In some examples, the system 500 may be comprised as a single device, for example a smartphone which is configured to provide stimuli by way of e.g. audio output, process the received inputs, and receive response inputs by way of a user interacting with the smartphone, e.g. by touching the touch sensitive screen of the smartphone. Other configurations may be envisaged.

In some examples, the apparatus 100 may be configured to further provide an ambient control signal to an ambient stimulus apparatus in accordance with a stored user profile. For example, the ambient stimulus apparatus may be a lamp or continuous light source, an audio output device, or a thermostat controlled heating or cooling device, for example. An ambient light source may be a source separate from a response means or may be part of the response means. The apparatus 100 may be configured to determine the ambient control signal further in accordance with the stimuli. As an example, if a user is receiving stimuli as part of a dynamic stimuli pattern to induce a waking state in the user, the apparatus may also provide an ambient control signal to a connected room light and/or thermostat to increase the level of ambient light and/or room temperature in accordance with the user's determined cognitive state of awakeness/awareness. As another example, if the user is using the apparatus 100 to induce sleep at dusk, then the speed at which the ambient light dims may be performed corresponding to any acceleration in the provision of stimuli according to the dynamic stimuli pattern due to the user's responses.

Light composition (wavelength, spectral power of each wavelength, light intensity) plays an important role in circadian biology, where blue light in the evening for example may be considered to be detrimental to a user falling sleep, and blue enriched lights may be used to treat winter depression, for example. Light also plays an important role in keeping a user's internal rhythms aligned with the environmental time. For example when suffering from jet-lag, the body needs to re-adjust to the new environmental light-dark schedule and this process can be speeded up or delayed by the provision of appropriate light at the right times.

Examples disclosed herein may provide ambient lighting, which is tailored to the particular user, and which is beneficial for the cognitive change of state the user wishes to achieve at a particular time of day. For example, for many users, an appropriate ambient lighting may comprise omitting blue light in the evening, with the emitted light of a low intensity but enough to read a book by; omitting blue and possibly also green light in the middle of the night and of an intensity as low as possible, but still enough to make out some broad features such that the user can find their way to the bathroom for example; and a dawn-appropriate light (e.g. simulating the colour spectrum of natural sunlight) which can aid the user to wake up, which changes to a more blue enriched light towards the end of the dawn period, and blue enriched light at time of a morning alarm sounding to wake the user.

For some users, however, a dramatically different schedule may be in place, either long-term or temporarily. People with delayed sleep phase syndrome may benefit from blue enriched light in their very early morning to have the biggest phase shifting effect, and this is at a time where the person is still asleep. Ambient lighting may be provided, therefore, which comprises blue enriched light at dawn for e.g. 10 minutes, so that the user does not have to set an alarm and turn on a lamp themselves, but they can open their eyes a little to check the lighting, and continue sleeping afterwards. For people with advanced sleep phase syndrome, they may benefit from blue enriched light at the beginning of their night (where this is advised against for the general population) and the ambient lighting provided for these users may be so tailored. Other examples of user-tailored ambient lighting which may be provided may be light which assists with helping a user overcome their jetlag quicker, with blue enriched light provided automatically either at begin of the night, or end of the night, depending on the country they have visited.

A difficulty with providing circadian lighting is not just having access to the right light colour composition, but also knowing at what time of day the users should be exposed to a certain composition. Examples of ambient lighting as disclosed herein which can emit a different light depending on the time the light source is turned on, or emit an automatic light schedule during sleep, may be provided. In some examples, ambient lighting may be provided according to personal information about the user, to guide the provision of light having a composition to complement the maintenance or change of cognitive state of the user. Such personal information may include, for example, the user's chronotype (the natural inclination of a particular user to wake and sleep at certain times of the day; for example as determined through a questionnaire), their work schedule (e.g. by accessing the user's work calendar and/or set wake-up alarm times), the user's location (e.g. using their postcode, GPS determined coordinates, longitude/latitude, etc) and/or specific environmental light, if they are likely to benefit from of a specific light intervention to enhance the effectiveness of the stimulus-response activity according to the dynamic stimuli pattern.

FIG. 6a shows an example data set of stimuli 600 (a stimuli program) provided over a time period 606 before any adaptation in response to provided user responses. An apparatus 100 as described above may be used to provide control signals to a stimuli means according to the stimuli pattern 600. In other words, in this example, a control signal to control the stimulus means is made according to a stimuli pattern 600 but without any dependence on any characteristic of a previous response signal (the stimuli pattern in this example may be considered to be “static” as opposed to dynamic since there is no adaptation in response to a user's responses). This example stimuli pattern may be used, for example, to try and induce a user to relax and possibly fall asleep. The data set 600 shows a plot of stimuli magnitude 604 against time 602. It can be seen there are characteristics of the stimuli pattern which are described in relation to FIG. 3a, namely there are four stimuli phases (Phase 1 602a, Phase 2 602b, Phase 3 602c, and Phase 4 602d; note in other examples there may be a different number than four phases); the overall magnitude of the stimuli decreases as a particular phase progresses in time; the start of a subsequent phase has stimuli of magnitudes which are higher than the magnitude of stimuli at the end of the preceding phase; and the stimuli within a phase vary between upper and lower boundary magnitudes 616 as illustrated by the triangle-shapes boundaries 316 in FIG. 3a.

FIG. 6b shows an example data set of stimuli 650 provided over a time period 656 including adaptation of the stimuli provided in response to provided user responses. An apparatus 100 as described above may be used to provide control signals to a stimuli means according to the stimuli pattern 600 and according to user responses to provided stimuli. In other words, in this example, a control signal to control the stimulus means is provided by an apparatus 100 according to a dynamic stimuli pattern 650 as well as in dependence on a characteristic of a previous response signal. The dynamic stimuli pattern 650 may be considered “dynamic” as it has been adapted to accommodate the user's actual cognitive state as indicated by the characteristics of the user's responses. In this example the user responses indicate that the user is falling asleep as the program of stimuli is provided, so the apparatus adapts the dynamic stimuli pattern 650, compared with an un-adapted stimuli pattern 600, to accelerate the provision of later stimuli in the dynamic stimuli pattern 650. Thus the magnitude 604 of the stimuli in the dynamic stimuli pattern 650 falls more quickly than the statis stimuli pattern 600. Also the length of time for each phase 652a-d of the dynamic stimuli pattern 650 is shortened until the final phase 652d, which is maintained until the end of the stimuli pattern time period 656, but during which less frequent stimuli of lower magnitude are provided compared with an un-adapted stimuli pattern 600. Further, in this example, the range of magnitudes of stimuli provided in each phase 652a-d is smaller in the adapted dynamic stimuli pattern 650 than in the un-adapted stimulus pattern 600. This is because the user is determined by the apparatus 100 to be reacting well to the provided stimuli (as determined by the user responses indicative of the user relaxing and falling asleep), and so the user's attention does not need to be maintained to as great an extent through provision of a wider ranging magnitude of stimuli.

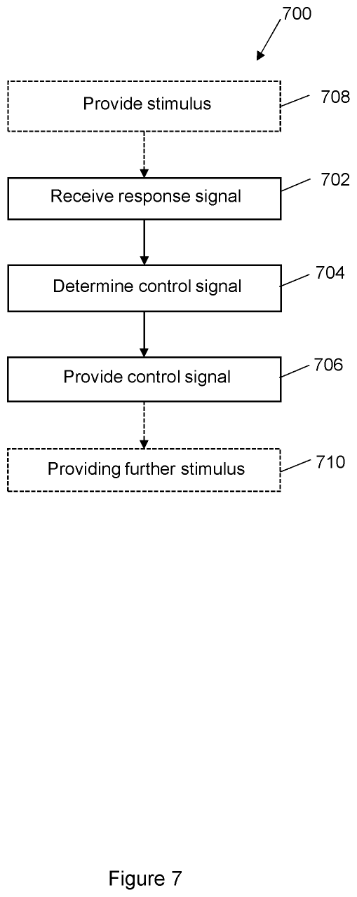

FIG. 7 shows an example computer-implemented method 700 for one or more of measuring cognitive state of a user and inducing a change of cognitive state of a user. The method may be performed by an apparatus 100 or system 500 as described above. The method comprises: receiving an response signal indicative of an active user response provided in response to a stimulus provided by a stimulus means 702; determining a control signal in dependence on a characteristic of the response signal and in accordance with a dynamic stimuli pattern, wherein the dynamic stimuli pattern is indicative of possible stimuli to be provided in response to the active user response 704; and providing the control signal to the stimulus means to control the stimulus means to provide a further stimulus in accordance with the control signal 706. The method may further comprise providing the stimulus by the stimulus means 708; and providing the further stimulus by the stimulus means in accordance with the control signal 710. Steps 708 and 710 may be performed by a stimulus means in communication with the apparatus 100.

In another aspect there is provided computer software which, when executed on a processor of any apparatus disclosed herein, or any system disclosed herein, is arranged to perform any method disclosed herein. In another aspect there is provided a non-transitory, computer-readable storage medium storing instructions thereon that, when executed by one or more electronic processors of any apparatus disclosed herein, or of any system disclosed herein, causes the one or more electronic processors to carry out any method disclosed herein.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine readable storage storing such a program. Still further, embodiments of the present invention may be conveyed electronically via any medium such as a communication signal carried over a wired or wireless connection and embodiments suitably encompass the same.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

1. An apparatus for one or more of measuring cognitive state of a user and inducing a change of cognitive state of a user, the apparatus configured to:
receive a response signal indicative of an active user response provided in response to a stimulus provided by a stimulus means;
determine a control signal in dependence on a characteristic of the response signal and in accordance with a dynamic stimuli pattern, wherein the dynamic stimuli pattern is indicative of possible stimuli to be provided in response to the active user response; and
provide the control signal to the stimulus means to control the stimulus means to provide a further stimulus in accordance with the control signal.

2. The apparatus of claim 1, further configured to:
receive a further response signal indicative of a further active user response provided in response to the further stimulus; and
determine a further control signal in dependence on a characteristic of the first and further response signals and in accordance with the dynamic stimuli pattern.

3. The apparatus of any preceding claim, wherein the characteristic of the response signal comprises one or more of:
a time delay between the provision of the stimulus and the receipt of the response signal in response to the stimulus;
a duration of provision of the active user response;
a pressure applied to provide the active user response;
a speed of provision of the active user response;
a determined user position during provision of the active user response; and
a volume of the active user response when the active user response is a sound.

4. The apparatus of any preceding claim, wherein the dynamic stimuli pattern comprises a reduction in one or more of stimulus amplitude and stimulus frequency as a function of time, wherein the rate of the reduction of the one or more of stimulus amplitude and stimulus frequency is dependent on the characteristics of the response signal.

5. The apparatus of any preceding claim, wherein the dynamic stimuli pattern comprises an increase in one or more of stimulus amplitude and stimulus frequency as a function of time, wherein the rate of the increase of the one or more of stimulus amplitude and stimulus frequency is dependent on the characteristics of the response signal.

6. The apparatus of claim 4 or claim 5, wherein the rate of the one or more of stimulus amplitude and stimulus frequency is further dependent on a passive user status signal received from a biosensor.

7. The apparatus of any preceding claim, wherein the dynamic stimuli pattern comprises a plurality of stimulus periods,
each of the stimulus periods is indicative of stimuli within a predetermined stimulus parameter range,
the predetermined stimulus parameter range of each stimulus period is at least partially different from the predetermined stimulus parameter range of each other stimulus period, and
the control signal is determined according to a subsequent stimulus period once an advance criterion of a current stimulus period is met as determined according to the characteristic of the response signal

8. The apparatus of claim 7, wherein the dynamic stimuli pattern further comprises a rest period between consecutive stimulus periods, and wherein, during the rest period, no stimuli are provided.

9. The apparatus of claim 7 or claim 8, wherein the predetermined stimulus parameter range indicates one or more of:
light stimuli within a predetermined amplitude range;
light stimuli within a predetermined wavelength range;
light stimuli within a predetermined frequency range;
light stimuli within a predetermined lumen range;
haptic stimuli within a predetermined amplitude range;
haptic stimuli within a predetermined duration range;
haptic stimuli within a predetermined duty range;
audio stimuli within a predetermined volume range;
audio stimuli within a predetermined pitch range;
audio stimuli within a predetermined duration range; and
a number of stimuli concurrently output as a stimulus group.

10. The apparatus of any of claims 7 to 9, wherein the advance criterion of a current stimulus period comprises one or more of:
an active user response reaction time being outside a predetermined reaction time period;
a number of active user responses being absent following a stimulus;
when the active user response is provided via application of pressure to a pressure sensor, an active user response having a pressure below a predetermined pressure threshold;
when the active user response is provided via provision of audio to an audio sensor, an active user response having a volume below a predetermined volume threshold; and
when an incorrect active user response is provided in response to a cognitive task stimulus.

11. The apparatus of any of claims 7 to 10, wherein the advance criterion of a current stimulus period comprises one or more of:
an active user response reaction time being within a predetermined reaction time period;
a threshold number of active user responses being made following respective stimuli;
when the active user response is provided via application of pressure to a pressure sensor, an active user response having a pressure above a predetermined pressure threshold;
when the active user response is provided via provision of audio to an audio sensor, an active user response having a volume above a predetermined volume threshold; and
when an correct active user response is provided in response to a cognitive task stimulus.

12. The apparatus of any of claims 7 to 11, wherein the control signal is further determined according to a subsequent stimulus period once a passive user advance criterion is met as determined according to a passive user status signal received from a biosensor.

13. The apparatus of any of claims 7 to 12, wherein the control signal is further determined according to a subsequent stimulus period when a duration for which a current stimulus period is in use reaches a current stimulus period threshold.

14. The apparatus of any preceding claim, wherein the dynamic stimuli pattern is indicative of a plurality of possible stimuli within a predetermined amplitude range; and one or more spike stimuli having an amplitude outside the predetermined amplitude range; and wherein the apparatus is configured to:
determine the control signal in accordance with the one or more spike stimuli if the characteristic of the response signal is indicative of an active user response outside a predetermined response range.

15. The apparatus of any preceding claim, wherein the apparatus is configured to:
determine that the user is asleep in dependence on a characteristic of one or more response signals; and
stop the provision of stimuli in dependence on determining that the user is asleep.

16. The apparatus of any preceding claim, wherein the apparatus is configured to:
determine that the user is asleep in dependence on a characteristic of one or more response signals;
pause the dynamic stimuli pattern to pause the provision of stimuli in dependence on determining that the user is asleep;
determine that the user is awake in dependence on receipt of a passive user status signal received from a biosensor;
if the user is determined to re-awaken within a predetermined period of time following the pause in provision of stimuli, recommence the provision of stimuli according to the paused dynamic stimuli pattern; and
if the user is determined to re-awaken after a predetermined period of time following the pause in provision of stimuli, recommence the provision of stimuli according to the start of a dynamic stimuli pattern.

17. The apparatus of any preceding claim, wherein the apparatus is configured to store the characteristics of the response signals provided by the user in response to the stimuli in a user response log.

18. The apparatus of any preceding claim, wherein the apparatus is configured to:
receive a sleep pattern change signal indicative of a requested change in sleep pattern; and
determine the dynamic stimuli pattern in accordance with a recorded current sleep time indicative of a time at which a user currently goes to sleep, and the requested change in sleep pattern, to induce sleep at a different time to the current sleep time.

19. The apparatus of any preceding claim, wherein the apparatus is configured to further provide an ambient control signal to an ambient stimulus apparatus in accordance with a stored user profile.

20. The apparatus of any preceding claim, wherein the apparatus is a processor, a portable electronic device, a mobile telephone, a docking station, and a server.

21. A system for one or more of measuring cognitive state of a user and inducing a change of cognitive state of a user, the system comprising:
the apparatus of any of claims 1 to 20; and
the stimulus means.

22. The system of claim 21, further comprising a response means configured to receive the active user response.

23. The system of claim 22, wherein the stimulus means comprises one or more of:
a light output device;
an image output device;
an audio output device; and
a haptic output device.

24. A computer-implemented method for one or more of measuring cognitive state of a user and inducing a change of cognitive state of a user, the method comprising:
receiving an response signal indicative of an active user response provided in response to a stimulus provided by a stimulus means;
determining a control signal in dependence on a characteristic of the response signal and in accordance with a dynamic stimuli pattern, wherein the dynamic stimuli pattern is indicative of possible stimuli to be provided in response to the active user response; and
providing the control signal to the stimulus means to control the stimulus means to provide a further stimulus in accordance with the control signal.

25. Computer software which, when executed on a processor of an apparatus according to any of claims 1 to 20 or a system according to any of claims 21-23, is arranged to perform a method according to claim 24.
